(19) United States
(12) Patent Application Publication
(10) Pub. No.: US 2024/0390561 A1
(43) Pub. Date: Nov. 28, 2024

(54) MEDICAL COATING AGENT AND MEDICAL DEVICE

(71) Applicants: TOAGOSEI CO., LTD., Tokyo (JP); KYUSHU UNIVERSITY, NATIONAL UNIVERSITY CORPORATION, Fukuoka-shi, Fukuoka (JP)

(72) Inventors: Masaru TANAKA, Fukuoka-shi, Fukuoka (JP); Shota TANIGUCHI, Nagoya-shi, Aichi (JP); Kenichi NAKAMURA, Nagoya-shi, Aichi (JP)

(73) Assignees: TOAGOSEI CO., LTD., Tokyo (JP); KYUSHU UNIVERSITY, NATIONAL UNIVERSITY CORPORATION, Fukuoka-shi, Fukuoka (JP)

(21) Appl. No.: 18/696,552

(22) PCT Filed: Sep. 28, 2022
(86) PCT No.: PCT/JP2022/036236

§ 371 Date: Mar. 28, 2024

(30) Foreign Application Priority Data
Sep. 30, 2021 (JP) 2021-161734

(51) Int. Cl. A61L 29/14; A61L 29/08; A61L 31/10; A61L 31/14

(52) U.S. Cl. CPC A61L 29/14; A61L 29/08; A61L 31/10; A61L 31/14

(57) ABSTRACT

A medical coating agent contains a polymer including a structural unit (A) derived from an ethylenically unsaturated monomer having a urethane bond. The polymer has a glass transition temperature of −25° C. or lower in a saturated hydration state, the state being defined as such a hydration state of the polymer that the top of the endothermic peak attributed to melting of ice emerges at 0° C. in a DSC curve obtained when the polymer which has been hydrated is heated by means of a differential scanning calorimeter (DSC) at a rate of 5° C./minute.

### TECHNICAL FIELD

### Cross-Reference to Related Applications

The present application claims the benefit of Japanese Patent Application No. 2021-161734 filed on Sep. 30, 2021, the disclosure of which is incorporated herein by reference.

The present disclosure relates to a medical coating agent and to a medical device. More particularly, the disclosure relates to a technique for imparting biocompatibility to a medical device which during use comes into contact with a biocomponent or a biotissue.

### Background Art

Medical devices are made from a variety of materials such as synthetic polymer, ceramic, glass, and metal. When such a medical device comes into contact with a biocomponent or a biotissue of a living body, the living body recognizes the medical device as a foreign object, whereby the function of the medical device may be impeded, or the living body may be adversely affected. For example, when a medical device is in contact with blood during use thereof, the medical device is recognized as a foreign object, whereby a bioprotective function may be activated to form thrombi. Thus, hitherto, attention has been paid to imparting biocompatibility to a medical device by use of a biocompatible synthetic polymer (see, for example, Patent Document 1).

Patent Document 1 discloses use of a polymer including a structural unit derived from 2-methoxyethyl acrylate as a biocompatible medical material.

### PRIOR ART DOCUMENTS

### Patent Documents

Patent Document 1: Japanese Patent Application Laid-Open (kokai) No. H04-152952

### SUMMARY OF THE INVENTION

### Problems to be Solved by the Invention

The main blood components involved in thrombogenesis are conceived to be thrombocytes (blood platelets) and fibrinogen. The present inventors have thought that inhibition of recognition of these components as foreign bodies by a medical device inserted in the living body is important. In other words, if the surface of a substrate of a medical device can be fully provided with an inhibitory property to adsorption of thrombocytes and fibrinogen (hereinafter may also be referred to as “anti-adsorption property”), the medical device can be provided with excellent anti-thrombogenicity, to thereby improve biocompatibility.

The present disclosure has been made under such circumstances, and an object of the disclosure is to provide a medical coating agent which exhibits high anti-adsorption property with respect to thrombocytes and fibrinogen and excellent anti-thrombogenicity.

### Means for Solving the Problems

The present disclosure provides the following means.
[1] A medical coating agent which contains a polymer including a structural unit (A) derived from an ethylenically unsaturated monomer having a urethane bond, wherein the polymer has a glass transition temperature of −25° C. or lower in a saturated hydration state, the state being defined as such a hydration state of the polymer that the top of the endothermic peak attributed to melting of ice emerges at 0° C. in a DSC curve obtained when the polymer which has been hydrated is heated by means of a differential scanning calorimeter (DSC) at a rate of 5° C./minute.
[2] The medical coating agent according to [1] above, wherein the polymer includes the structural unit (A) in an amount of 10 mass % or more with respect to the entire structural units included in the polymer.
[3] The medical coating agent according to [1] or [2] above, wherein the polymer exhibits a difference in glass transition temperature of 25° C. or greater between a dry state and a saturated hydration state, the glass transition temperatures being measured by means of a differential scanning calorimeter at a heating (i.e., temperature elevation) rate of 5° C./minute.
[4] The medical coating agent according to any of [1] to [3] above, wherein the polymer is a (meth)acrylic polymer.
[5] A medical device having a substrate coated with a medical coating agent according to any of [1] to [4] above.

### Advantageous Effects of the Invention

The polymer contained in the medical coating agent of the present disclosure exhibits high anti-adsorption property with respect to thrombocytes and fibrinogen and excellent anti-thrombogenicity. Thus, by coating a substrate of a medical device with the medical coating agent of the present disclosure, the provided medical device can achieve excellent anti-thrombogenicity.

### MODES FOR CARRYING OUT THE INVENTION

The present disclosure will next be described in detail. In the present specification, the expression “(meth)acrylic” refers to “acrylic and/or methacrylic”. Similarly, “(meth)acrylate” refers to “acrylate and/or methacrylate”.

### <<Medical Coating Agent>>

The medical coating agent of the present disclosure contains a polymer including structural unit (A) derived from an ethylenically unsaturated monomer having a urethane bond (—NH—COO—), and having a glass transition temperature of −25° C. or lower in a saturated hydrate state (hereinafter, the polymer may also be referred to as “polymer (P)”).

When water is incorporated into polymer (P), water interacting with polymer (P) (i.e., hydration water) may exist in three forms; “free water”, “non-freezing water”, and “intermediate water”, depending on the intensity of interaction with the polymer. Among these three water forms, “free water” refers to a water form which weakly interacts with the polymer and has a freezing point of 0° C. “Non-freezing water” refers to a water form which strongly interacts with the polymer and exhibits no detectable freezing point. “Intermediate water” refers to a water form which exhibits a strength of interaction with the polymer between that of free water and that of non-freezing water (i.e., which exhibits relatively mild interaction with the polymer) and has a freezing point lower than 0° C. Acquisition of biocompatibility by a polymer conceivably relates to a large amount of intermediate water being contained in the polymer in a hydrated state (see, for example, paragraphs 0003 and 0004 of Japanese Patent Application Laid-Open (kokai) No. 2016-35000).

More specifically, in the living body, a bioprotective function is activated through recognition of a foreign object by cells, to thereby evoke rejection. Accordingly, when a medical device is in contact with a biocomponent or a biotissue in the case of medical treatment, surgery, or the like, the living body may possibly recognize the medical device as a foreign object. If the bioprotective function is activated in such a case, the therapeutic operation or the like may be impeded. In one conceivable case, when a medical device comes into contact with blood, thrombosis occurs, whereby the target function of the medical device may be impeded, or the living body may be adversely affected. In contrast, conceivably, the living body has difficulty in recognizing, as a foreign object, a polymer body retaining intermediate water in a surface portion, to thereby leading to excellent anti-thrombogenicity.

Meanwhile, thrombocytes and fibrinogen are known to be blood components involved in formation of thrombi. Thrombocytes are blood cells which are activated by a foreign object and which coagulate on the foreign object, to thereby form thrombi. Thrombocytes are involved in primary hemostasis in a hemostatic process. Fibrinogen, serving as blood coagulation factor I, is a protein which is converted to fibrin in the final stage of blood clotting, to thereby form coagulation thrombi. Fibrinogen is involved in secondary hemostasis in a hemostatic process. That is, both thrombocytes and fibrinogen are main components involved in thrombosis. It is conceivably important for biocompatibility (more specifically, anti-thrombogenicity) that adsorption of these components on the polymer is prevented.

In this respect, the polymer (P) contained in the medical coating agent of the present disclosure readily achieves retention of intermediate water in hydration, and can satisfactorily prevent adsorption of thrombocytes and fibrinogen on the surface of the substrate coated with the polymer (P). As a result, excellent anti-thrombogenicity may be achieved.

In the present specification, the “saturated hydration state” is defined as such a hydration state of the polymer that the top of the endothermic peak attributed to melting of ice emerges at 0° C. in a DSC curve obtained when the polymer which has been hydrated is heated at a rate of 5° C./minute. However, in the expression found in the present specification “the top of the endothermic peak attributed to melting of ice emerges at 0° C.,” an error about the temperature of 0° C. at which the top of the endothermic peak emerges is allowed (e.g., 0° C.±0.2° C.), so long as the polymer contains a sufficient amount of water (i.e., the polymer is considered to be in a saturated hydration state).

Hereinafter, the ingredients contained in the medical coating agent of the present disclosure will be described in detail.

### <Polymer (P)>

The polymer (P) includes a structural unit derived from an ethylenically unsaturated monomer having a urethane bond (hereinafter may also be referred to as “monomer (M)”).

From the viewpoints of easy enhancement of monomer reactivity and easy industrial production, the polymer (P) is preferably a (meth)acrylic polymer. Specifically, the amount of structural units derived from a (meth)acrylic monomer with respect to the entire structural units derived from the monomer(s) forming the polymer (P) is preferably more than 50 mass %, more preferably 60 mass % or more, still more preferably 70 mass % or more, yet more preferably 80 mass % or more, further more preferably 90 mass % or more.

The monomer (M) is preferably a compound that can incorporate a moiety having a urethane bond into side chains of the polymer. Particularly, a (meth)acrylic monomer having a urethane bond is preferred. The case where the monomer (M) is a (meth)acrylic monomer is preferred, since the monomer reactivity can be easily enhanced. Such monomers (M) may be used singly or in combination of two or more species.

From the viewpoint of sufficiently lowering the glass transition temperature (Tg2) of the polymer (P) in a saturated hydration state, there is preferably used as the monomer (M) (methoxycarbonyl)aminoalkyl (meth)acrylate or a compound represented by the following formula (I):

CH₂═CR¹—COO—R²—NH—COO—R³—R⁴ (I)

(wherein R¹ represents a hydrogen atom or a methyl group; R² represents a C1 to C5 alkylene group or a group represented by “—(R⁵O)ₘ—R⁶—” (wherein R⁵ represents a C1 to C3 alkylene group; R⁶ represents a C1 to C3 alkylene group; and m is an integer of 1 to 3); R³ represents a C1 to C3 alkylene group; any hydrogen atom present in the alkylene group may be substituted by a C1 to C10 alkoxy group; and R⁴ is a C1 to C10 alkoxy group).

Further, from the viewpoint of sufficiently enhancing anti-adsorption property with respect to thrombocytes and fibrinogen, regarding R³ in formula (I), any hydrogen atom present in the alkylene group is preferably substituted by a C1 to C4 alkoxy group, more preferably substituted by a C1 to C2 alkoxy group. From the same viewpoint, R⁴ in formula (I) is preferably a C1 to C4 alkoxy group, more preferably C1 to C2 alkoxy group.

Specific examples of the (methoxycarbonyl)aminoalkyl (meth)acrylate include (methoxycarbonyl)aminomethyl (meth)acrylate, 2-((methoxycarbonyl)amino) ethyl (meth)acrylate, and 3-((methoxycarbonyl)amino) propyl (meth)acrylate. Among them, from the viewpoint of sufficiently lowering the glass transition temperature (Tg2) of the polymer (P) in a saturated hydration state, 2-((methoxycarbonyl)amino) ethyl acrylate is preferably used.

Specific examples of the compound represented by the aforementioned formula (I) include 2-(((2-methoxyethoxy)carbonyl)amino)ethyl (meth)acrylate, 2-(((2-ethoxyethoxy) carbonyl)amino)ethyl (meth)acrylate, 2-(((2-propoxyethoxy) carbonyl)amino)ethyl (meth)acrylate, 2-((((1,3-dimethoxypropan-2-yl)oxy)carbonyl)amino)ethyl (meth)acrylate, 2-((((1,3-diethoxyprropan-2-yl)oxy)carbonyl)amino)ethyl (meth)acrylate, 2-((((1-methoxy-3-ethoxyprppan-2-yl)oxy)carbonyl)amino)ethyl (meth)acrylate, 6-oxo-2,5,10-trioxa-7-azadodecan-12-yl (meth)acrylate, and 7-oxo-3,6,11-trioxa-8-azatridecan-13-yl (meth)acrylate.

From the viewpoints of incorporating a sufficient amount of a side chain structure having a urethane bond into the polymer and sufficiently lowering the glass transition temperature (Tg2) of the polymer (P) in a saturated hydration state, the polymer (P) preferably includes a structural unit derived from at least one compound selected from the group consisting of (methoxycarbonyl)aminoalkyl (meth)acrylate and the compounds represented by the aforementioned formula (I) (hereinafter may also be referred to as “monomer (m−1)”) in an amount of 10 mass % or more, with respect to the all the structural units derived from the monomer(s) forming the polymer (P), more preferably 20 mass % or more, still more preferably 30 mass % or more, yet more preferably 50 mass % or more, further more preferably 60 mass % or more. The polymer (P) may include structural units derived from the monomer (m−1) singly or in combination of two or more species.

The polymer (P) may be formed only of a structure unit derived from the monomer (m−1). Alternatively, the polymer (P) may further include a structural unit derived from a monomer other than the monomer (m−1) (hereinafter may also be referred to as an “additional monomer”) for the purpose of, for example, controlling the glass transition temperature of the polymer, so long as the effects of the present disclosure are not impaired.

Examples of the additional monomer include monomers which have no urethane bond and which are co-polymerizable with the monomer (m−1). Examples of such a monomer include an unsaturated carboxylic acid, an unsaturated acid anhydride, an alkyl (meth)acrylate ester, an alicyclic (meth)acrylate ester, an aromatic (meth)acrylate ester, an alkoxyalkyl (meth)acrylate ester, a hydroxyalkyl (meth)acrylate ester, a polyalkylene glycol mono(meth)acrylate, a vinyl compound having a heterocyclic structure, a vinyl compound having an amino group, a vinyl compound having an amido group, a vinyl compound having a nitrile group, an aromatic vinyl compound, and a maleimide compound.

Specific examples of the unsaturated carboxylic acid include (meth)acrylic acid, itaconic acid, crotonic acid, maleic acid, fumaric acid, citraconic acid, cinnamic acid, succinic acid monohydroxyethyl (meth)acrylate, ω-carboxy-caprolactone mono (meth)acrylate, β-carboxyethyl (meth)acrylate, and 4-carboxystyrene.

Examples of the unsaturated acid anhydride include maleic anhydride, itaconic anhydride, and citraconic anhydride.

Examples of the alkyl (meth)acrylate ester include methyl (meth)acrylate, ethyl (meth)acrylate, isopropyl (meth)acrylate, n-propyl (meth)acrylate, n-butyl (meth)acrylate, isobutyl (meth)acrylate, tert-butyl (meth)acrylate, hexyl (meth)acrylate, and 2-ethylhexyl (meth)acrylate.

Specific examples of the alicyclic (meth)acrylate ester include cyclohexyl (meth)acrylate, methylcyclohexyl (meth)acrylate, tert-butylcyclohexyl (meth)acrylate, cyclododecyl (meth)acrylate, isobornyl (meth)acrylate, adamantyl (meth)acrylate, dicyclopentenyl (meth)acrylate, and dicyclopentanyl (meth)acrylate. Specific examples of the aromatic (meth)acrylate ester include phenyl (meth)acrylate, benzyl (meth)acrylate, phenoxymethyl (meth)acrylate, 2-phenoxyethyl (meth)acrylate, and 3-phenoxypropyl (meth)acrylate.

Specific examples of the alkoxyalkyl (meth)acrylate ester include 2-methoxyethyl (meth)acrylate, 2-ethoxyethyl (meth)acrylate, n-propoxyethyl (meth)acrylate, n-butoxyethyl (meth)acrylate, 3-methoxypropyl (meth)acrylate, 3-ethoxypropyl (meth)acrylate, n-propoxypropyl (meth)acrylate, n-butoxypropyl (meth)acrylate, methoxybutyl (meth)acrylate, ethoxybutyl (meth)acrylate, n-propoxybutyl (meth)acrylate, and n-butoxybutyl (meth)acrylate.

Specific examples of the hydroxyalkyl (meth)acrylate ester include 2-hydroxyethyl (meth)acrylate, 2-hydroxypropyl (meth)acrylate, 3-hydroxypropyl (meth)acrylate, 2-hydroxybutyl (meth)acrylate, 3-hydroxybutyl (meth)acrylate, and 4-hydroxybutyl (meth)acrylate. Examples of the polyalkylene glycol mono (meth)acrylate include polyethylene glycol mono (meth)acrylate, polypropylene glycol mono (meth)acrylate, and polyethylene glycol-polypropylene glycol mono (meth)acrylate.

Examples of the vinyl compound having a heterocyclic structure include glycidyl (meth)acrylate, (3,4-epoxycyclohexyl)methyl (meth)acrylate, and tetrahydrofurfuryl (meth)acrylate.

Examples of the vinyl compound having an amino group include dimethylaminomethyl (meth)acrylate, diethylaminomethyl (meth)acrylate, 2-dimethylaminoethyl (meth)acrylate, 2-diethylaminoethyl (meth)acrylate, 2-(di-n-propylamino)ethyl (meth)acrylate, 2-dimethylaminopropyl (meth)acrylate, 2-diethylaminopropyl (meth)acrylate, 2-(di-n-propylamino) propyl (meth)acrylate, 3-dimethylaminopropyl (meth)acrylate, 3-diethylaminopropyl (meth)acrylate, and 3-(di-n-propylamino) propyl (meth)acrylate.

Examples of the vinyl compound having an amido group include (meth) acrylamide, N, N-dimethyl (meth) acrylamide, N, N-dimethylaminopropyl (meth) acrylamide, and N-methylol (meth) acrylamide.

Examples of the vinyl compound having a nitrile group include cyanomethyl (meth)acrylate, 1-cyanoethyl (meth)acrylate, 2-cyanoethyl (meth)acrylate, 1-cyanopropyl (meth)acrylate, 2-cyanopropyl (meth)acrylate, 3-cyanopropyl (meth)acrylate, 4-cyanobutyl (meth)acrylate, 6-cyanohexyl (meth)acrylate, 2-ethyl-6-cyanohexyl (meth)acrylate, 8-cyanooctyl (meth)acrylate, (meth) acrylonitrile, α-ethylacrylonitrile, α-isopropylacrylonitrile, α-chloroacrylonitrile, and α-fluoroacrylonitrile.

Examples of the aromatic vinyl compound include styrene, α-methylstyrene, β-methylstyrene, vinylxylene, methylstyrene, ethylstyrene, butylstyrene, methoxystyrene, hydroxystyrene, isopropenylphenol, vinyl benzoate, and vinylnaphthalene.

Examples of the maleimide compound include a maleimide compound and an N-substituted maleimide compound. Examples of the N-substituted maleimide compound include N-alkyl-substituted maleimides such as N-methylmaleimide, N-ethylmaleimide, N-n-propylmaleimide, N-isopropylmaleimide, N-n-butylmaleimide, N-isobutylmaleimide, and N-tert-butylmaleimide; N-cycloalkyl-substituted maleimides such as N-cyclopentylmaleimide and N-cyclohexylmaleimide; N-aralkyl-substituted maleimides such as N-benzylmaleimide; and N-aryl-substituted maleimides such as N-phenylmaleimide, N-(4-hydroxyphenyl) maleimide, N-(4-acetylphenyl) maleimide, and N-(4-methoxyphenyl) maleimide.

In addition to the aforementioned monomers, a monomer which has a urethane bond and which is co-polymerizable with the monomer (m−1) may also be used as the additional monomer. Examples of such a monomer include 2-((ethoxycarbonyl)amino)ethyl (meth)acrylate and 2-((isopropoxycarbonyl)amino)ethyl acrylate. The additional monomer may be used singly or in combination of two or more species.

From the viewpoint of producing a polymer having no water solubility and high biocompatibility, among the aforementioned monomers, the additional monomer is preferably at least one species selected from the group consisting of an alkyl (meth)acrylate ester, an alicyclic (meth)acrylate ester, an aromatic (meth)acrylate ester, an alkoxyalkyl (meth)acrylate ester, a vinyl compound having an amino group, and a vinyl compound having an amido group, more preferably at least one species selected from the group consisting of an alkyl (meth)acrylate ester and an alkoxyalkyl (meth)acrylate ester.

Of these, the additional monomer is preferably at least one species selected from the group consisting of alkyl (meth)acrylate ester having a C1 to C12 alkyl group and an alkoxyalkyl (meth)acrylate ester having a C3 to C12 alkoxyalkyl group, more preferably an alkoxyalkyl (meth)acrylate ester having a C3 to C12 alkoxyalkyl group, still more preferably an alkoxyalkyl (meth)acrylate ester having a C3 to C4 alkoxyalkyl group, particularly preferably 2-methoxyethyl (meth)acrylate. By forming the polymer (P) from one or more members of the above compounds, a coating agent which can effectively suppress adsorption of thrombocytes and fibrinogen can be provided.

When the polymer (P) includes a structural unit derived from at least one monomer selected from the group consisting of alkyl (meth)acrylate ester having a C1 to C12 alkyl group and an alkoxyalkyl (meth)acrylate ester having a C3 to C12 alkoxyalkyl group (hereinafter may also be referred to as a “monomer (N)”), the amount of the structural unit derived from the monomer (N) with respect to the entire structural units derived from monomers forming the polymer (P) is preferably 5 mass % or more, more preferably 10 mass % or more, still more preferably 20 mass % or more. From the viewpoint of reducing a drop in performance attributed to an insufficient amount of incorporated structural units (A), the upper limit of the amount of the structural unit derived from the monomer (N) with respect to the entire structural units derived from monomers forming the polymer (P) is preferably 90 mass % or less, more preferably 80 mass % or less, still more preferably 70 mass % or less, yet more preferably 50 mass % or less, further more preferably 40 mass % or less.

Preferably, the polymer (P) include the structural unit (A) in an amount of 10 mass % or more with respect to the entire structural units included in the polymer (P). When the amount of structural unit (A) in the polymer (P) falls within the aforementioned range, there can be satisfactorily provided an effect of suppressing adsorption of thrombocytes and fibrinogen on a substrate coated with the medical coating agent of the present disclosure, to thereby yield a medical device having excellent anti-thrombogenicity, which is preferred. From this viewpoint, the amount of structural unit (A) with respect to the entire structural units included in the polymer (P) is preferably 20 mass % or more, more preferably 30 mass % or more, still more preferably 50 mass % or more, yet more preferably 60 mass % or more.

When the polymer (P) is a copolymer of the monomer (M) and an additional monomer having no urethane bond, the polymer (P) may be any of a random copolymer, a block copolymer, a graft copolymer, and the like. From the viewpoint of enhancing the effect of improving anti-thrombogenicity by uniformly incorporating a structure having a urethane bond into the entirety of the polymer, the polymer (P) is preferably a random copolymer.

The weight average molecular weight (Mw) of the polymer (P) is preferably 2,000 to 2,000,000. When Mw is 2,000 or higher, a sufficient mechanical strength of the coating layer formed by use of the medical coating agent can be secured. Also, when Mw is 2,000,000 or lower, an excessive rise in viscosity of the medical coating agent can be suppressed, whereby coatability and handing property can be secured. The Mw of the polymer (P) is more preferably 5,000 or higher, still more preferably 10, 000 or higher, yet more preferably 30,000 or higher, further more preferably 50, 000n or higher. The upper limit of the Mw of the polymer (P) is more preferably 1,500,000 or lower, still more preferably 1,000,000 or lower. In the present specification, Mw of the polymer is a molecular weight as reduced to polystyrene as a standard, obtained through gel permeation chromatography (GPC).

No particular limitation is imposed on the polymerization method for producing the polymer (P). Actually, the polymer (P) may be produced by polymerizing monomers through, for example, a known radical polymerization technique such as solution polymerization, suspension polymerization, emulsion polymerization, or bulk polymerization. In one possible mode of the solution polymerization, an organic solvent and monomers are fed to a reactor, and a polymerization initiator (e.g., an azo compound) is added thereto. The mixture is heated at 40 to 250° C. for polymerization, to thereby yield a target polymer. When the polymer obtained through polymerization reaction is subjected to isolation and/or purification, such a treatment may be conducted through a known method.

No particular limitation is imposed on the glass transition temperature (Tg1) of the polymer (P) in a dry state, and the Tg1 is, for example, 20° C. or lower, preferably 10° C. or lower. No particular limitation is imposed on the lower limit of the glass transition temperature (Tg1) of the polymer (P) in a dry state, and the lower limit is, for example, −70° C. or higher. In the present specification, the glass transition temperature of the polymer (P) is a value determined by means of a differential scanning calorimeter (DSC) at a heating rate of 5° C./minute. The specific procedure of the measurement is based on a corresponding method in the Examples mentioned below (the same is applicable to the glass transition temperature (Tg2) of the polymer in a saturated hydration state).

The polymer (P) has a glass transition temperature (Tg2) of −25° C. or lower in its saturated hydration state. When the glass transition temperature (Tg2) of the polymer (P) in a saturated hydration state is higher than −25° C., an anti-adsorption property with respect to thrombocytes and fibrinogen fails to be fully imparted to the surface of a substrate, whereby a target anti-thrombogenicity cannot possibly be imparted to the substrate. From the viewpoint of sufficiently enhancing the anti-adsorption property with respect to thrombocytes and fibrinogen, the glass transition temperature (Tg2) of the polymer (P) in a saturated hydration state is preferably −30° C. or lower, more preferably −40° C. or lower, still more preferably −50° C. or lower. No particular limitation is imposed on the lower limit of the glass transition temperature (Tg2) of the polymer (P) in a saturated hydration state, and the lower limit is, for example, −100° C. or higher.

The glass transition temperature (Tg2) of the polymer (P) in a saturated hydration state can be adjusted to fall within a desired temperature range by modifying the type and amount of the monomer(s) forming the polymer (P). For example, when the glass transition temperature, in a saturated hydration state, of a homopolymer which is formed of the monomer (M) used for producing the polymer (P) is higher than a target temperature, the glass transition temperature (Tg2) of the polymer (P) in its saturated hydration state can be modified by use of a monomer as a copolymerization component, which provides a glass transition temperature of the homopolymer in its saturated hydration state lower than that of the monomer (M).

Also, from the viewpoint of sufficiently enhancing the anti-adsorption property with respect to thrombocytes and fibrinogen, the difference between the glass transition temperature (Tg1) of the polymer (P) in a dry state and the glass transition temperature (Tg2) of the polymer (P) in a saturated hydration state; i.e., ΔTg (=Tg1−Tg2), is preferably 25° C. or more. The difference ΔTg is more preferably 30° C. or more, still more preferably 35° C. or more.

### <Optional Components (Ingredients)>

The medical coating agent of the present disclosure may further contain an ingredient other than the polymer (P) (hereinafter may be referred to as an “additional component”) according to the purpose of use and the like. When the medical coating agent of the present disclosure is in the form of liquid, one mode of the medical coating agent is a polymer composition containing the polymer (P) which is optionally dissolved or dispersed in a solvent.

When the medical coating agent of the present disclosure contains a solvent, a solvent which can dissolve the polymer (P) is preferably used as the solvent. The solvent contained in the medical coating agent of the present disclosure is preferably an organic solvent. Specific examples of the organic solvent include alcohols such as methanol, ethanol, n-propanol, and isopropanol; ketones such as acetone and methyl ethyl ketone; ethers such as ethylene glycol monomethyl ether, propylene glycol monomethyl ether, tetrahydrofuran, and dioxane; esters such as ethylene glycol monomethyl ether acetate and ethyl acetate; amide-based solvents such as N, N-dimethylformamide (DMF) and N, N-dimethylacetamide; hydrocarbons such as n-hexane, cyclohexane, toluene, and xylene; and dimethylsulfoxide. These solvents may be used singly or in combination of two or more species.

Examples of the additional component (other than solvent) which may be incorporated into the medical coating agent of the present disclosure include various drugs such as an antibacterial agent, an antiinflammatory agent, and an antioxidant. The additional component may be used singly or in combination of two or more species. The amount of the additional component may be tuned in accordance with the type of the component, so long as the effects of the present disclosure are not impaired.

In the medical coating agent of the present disclosure, the amount of the polymer (P) contained in the medical coating agent, with respect to the total amount (100 parts by mass) of the solid content (i.e., components except for the solvent contained in the medical coating agent), is preferably 50 parts by mass or more, more preferably 70 parts by mass or more, still more preferably 80 parts by mass or more, yet more preferably 90 parts by mass or more, further more preferably 95 parts by mass or more. Through adjusting the polymer (P) content to satisfy the above conditions, excellent anti-thrombogenicity is achieved, and consistent biocompatibility can be imparted to a substrate, which is preferred.

When the medical coating agent of the present disclosure is in the form of solution, no particular limitation is imposed on the solid content (i.e., a ratio of the mass of the components except for the solvent contained in the medical coating agent to the volume of the solvent used for preparing the medical coating agent) of the medical coating agent. However, the solid content is preferably 0.001 to 30 (w/v) %. By adjusting the solid content to 0.001 (w/v) % or more, a coating layer having sufficient thickness and mechanical strength can be formed on a substrate. When the solid content is 30 (w/v) % or less, suitable coatability can be secured, and a coating layer having a uniform thickness is easy to form. The solid content of the polymer composition is more preferably 0.01 to 25 (w/v) %, still more preferably 0.05 to 20 (w/v) %.

### <<Medical Devices>>

The medical device of the present disclosure includes a substrate coated with the aforementioned medical coating agent of the present disclosure. A part or the entirety of a surface of the medical device of the present disclosure is coated with the polymer (P) contained in the medical coating agent of the present disclosure. Thus, the device exhibits a high anti-adsorption property with respect to thrombocytes and fibrinogen and excellent anti-thrombogenicity.

No particular limitation is imposed on the substrate of the medical device to which the medical coating agent of the present disclosure is applied. Examples of the material of the substrate include various materials such as resin, rubber, metal, glass and ceramic. Examples of the resin include various resin materials such as polycarbonate, polyethylene terephthalate, polyvinyl chloride, polyethylene, polypropylene, polymethylpentene, polyurethane, poly(meth)acrylate, polystyrene, polyacetal, polysulfone, polyether sulfone, fluororesins (e.g., polyvinylidene fluoride and poly(tetrafluoroethylene), acrylonitrile-butadiene-styrene (ABS) resin, polyamide, and ethylnene-vinyl acetate-based resin. Examples of the rubber include silicone rubber and urethane rubber. Examples of the metal include stainless steel, titanium, and aluminum. The material forming the substrate of the medical device may be a mixture of two or more materials.

No particular limitation is imposed on the method of coating the substrate surface with the medical coating agent of the present disclosure. For example, when the medical coating agent is in the form of solution, the medical coating agent is applied to the surface of the substrate, and the solvent of the agent is removed by heating or other means. As a result, a medical device in which at least a part of the substrate surface is coated with the polymer (P) is produced.

The application method may be appropriately selected in accordance with the shape of the substrate, purpose of use, etc. Examples of the application method include various application means such as a bar coater, an applicator, a doctor blade, a dip coater, a roll coater, a spin coater, a flow coater, a knife coater, a comma coater, a reverse coater, a die coater, a lip coater, a gravure coater, a microgravure coater, and an ink-jet device. The amount of the medical coating agent to be applied may be appropriately tuned in accordance with the purpose of use, material, etc. of the medical device, such that the thickness of the coating layer formed from the medical coating agent falls within a target range.

No particular limitation is imposed on the medical device in which the surface of the substrate is to be coated with the medical coating agent of the present disclosure, and the medical coating agent can be applied to various medical devices. Specific examples of such medical devices include a stent, a catheter, a blood bag, infusion apparatus, surgical instruments, dental instruments, a blood circulator, a blood purifier, a plasma separator, an artificial blood vessel, and artificial organs (e.g., artificial heart-lung apparatus and artificial kidney apparatus). Also, in application of the medical coating agent of the present disclosure to a medical device, no particular limitation is imposed on the purpose and use of the application. For example, the medical coating agent of the present disclosure may be used as an anti-bacterial/anti-fouling coating agent. Since the medical coating agent of the present disclosure can impart excellent anti-thrombogenicity to the surface of a substrate, the medical coating agent of the present disclosure is preferably applied as a coating material for, among others, a substrate of a medical device used in direct contact with blood.

### EXAMPLES

The disclosure will be described more specifically by way of example, which should not be construed as limiting the disclosure thereto. Unless otherwise specified, the units “part(s)” and “%” refer to “part(s) by mass” and “mass %”, respectively.

### 1. Synthesis of Ethylenically Unsaturated Monomers

### Synthesis Example 1: Synthesis of 2-((Methoxycarbonyl)Amino) Ethyl Acrylate

To a 300-mL three-neck flask, a stirrer, tetrahydrofuran (product of FUJIFILM Wako Pure Chemical Corporation) (50 mL) serving as a solvent, dibutyltin dilaurate (product of FUJIFILM Wako Pure Chemical Corporation) (0.09 g) serving as a catalyst, and methanol (product of FUJIFILM Wako Pure Chemical Corporation) (2.64 g) serving as a raw material alcohol were added. A thermometer, a 50-mL dropping funnel, and a three-way cock were attached to the flask. Thereafter, 2-acryloyloxyethyl isocyanate (Karenz AOI, product of Showa Denko K.K., hereinafter may also be referred to as “AOI”) (10.58 g) was added to the dropping funnel.

Subsequently, nitrogen was caused to flow at 100 mL/min for 10 minutes through the three-way cock. After termination of nitrogen flow, a rubber balloon containing nitrogen was attached to the three-way cock, and the flask was cooled at 5° C. or lower on an ice bath. Then, while the inside temperature was maintained at 10° C. or lower, AOI was added dropwise through the dropping funnel. After termination of addition of AOI, the flask was heated to room temperature (25° C.), and stirring was continuously conducted overnight.

Thereafter, saturated aqueous sodium hydrogen carbonate (10 mL) was added dropwise to the flask, to thereby terminate reaction. The liquid contents (200 mL) of the flask were transferred to a separatory funnel, to thereby separate the liquid into an organic layer and an aqueous layer. Ethyl acetate (product of FUJIFILM Wako Pure Chemical Corporation, special grade) (30 mL) was added to the aqueous layer, and the mixture was shaken, to thereby effect extraction of the aqueous layer. Subsequently, the operation of shaking and extraction of the aqueous layer was further conducted twice. Separately, saturated brine (30 mL) was added to the recovered organic layer, and the mixture was shaken and subjected to washing. Subsequently, the operation of shaking and washing of the organic layer was further conducted twice. After completion of washing, sodium sulfate anhydrate (product of FUJIFILM Wako Pure Chemical Corporation) (30 mg) was added to the organic layer, and the mixture was stirred for 1 hour, to thereby dehydrate the mixture. The product was subjected to filtration with pleated filter paper, to thereby remove sodium sulfate. Then, p-methoxyphenol was added to the filtrate in such an amount that 250 ppm of the theoretic yield was attained.

Subsequently, the solvent of the resultant mixture was removed by means of a rotary evaporator, while the flask was immersed in a hot water bath at 40° C. After removal of the solvent, the product was allowed to stand in vacuum for 1 hour by means of a vacuum pump, to thereby further remove remaining solvent. The thus-recovered sample was purified through silica gel column chromatography using a mixture of hexane and ethyl acetate (1:1 by volume) as a solvent, to thereby yield 2-((methoxycarbonyl)amino) ethyl acrylate (hereinafter may also be referred to as “MOCNA”).

### Synthesis Example 2: Synthesis of 2-((Ethoxycarbonyl)Amino)Ethyl Acrylate

The procedure of Synthesis Example 1 was repeated, except that the raw material alcohol was changed to ethanol (product of FUJIFILM Wako Pure Chemical Corporation) (3.46 g), to thereby yield 2-((ethoxycarbonyl)amino)ethyl acrylate (hereinafter may also be referred to as “EOCNA”).

### Synthesis Example 3: Synthesis of 2-(((2-methoxyethoxy)carbonyl)amino)ethyl acrylate

The procedure of Synthesis Example 1 was repeated, except that the raw material alcohol was changed to 2-methoxyethanol (product of FUJIFILM Wako Pure Chemical Corporation) (6.28 g), to thereby yield 2-(((2-methoxyethoxy)carbonyl)amino)ethyl acrylate (hereinafter may also be referred to as “MEOCNA”).

### Synthesis Example 4: Synthesis of 2-((((1,3-dimethoxypropan-2-yl)oxy)carbonyl)amino)ethyl acrylate

The procedure of Synthesis Example 1 was repeated, except that the raw material alcohol was changed to 1,3-dimethoxy-2-propanol (product of Combi-Blocks) (9.91 g), to thereby yield 2-((((1,3-dimethoxypropan-2-yl)oxy)carbonyl)amino)ethyl acrylate (hereinafter may also be referred to as “DMPOCNA”).

### Synthesis Example 5: Synthesis of 2-((((1,3-diethoxypropan-2-yl)oxy)carbonyl)amino)ethyl acrylate

The procedure of Synthesis Example 1 was repeated, except that the raw material alcohol was changed to 1,3-diethoxy-2-propanol (product of Tokyo Chemical Industry Co., Ltd.) (9.91 g), to thereby yield 2-((((1,3-diethoxypropan-2-yl)oxy)carbonyl)amino)ethyl acrylate (hereinafter may also be referred to as “DEPOCNA”).

### Synthesis Example 6: Synthesis of 2-((isopropoxycarbonyl)amino)ethyl acrylate

The procedure of Synthesis Example 1 was repeated, except that the raw material alcohol was changed to 2-propanol (product of FUJIFILM Wako Pure Chemical Corporation) (4.96 g), to thereby yield 2-((isopropoxycarbonyl)amino)ethyl acrylate (hereinafter may also be referred to as “IPOCNA”).

### Synthesis Example 7: Synthesis of 6-oxo-2,5,10-trioxa-7-azadodecan-12-yl methacrylate

The procedure of Synthesis Example 1 was repeated, except that AOI was changed to 2-(2-methacryloyloxyethyloxy)ethyl isocyanate (Karenz MOI-EG, product of Showa Denko K.K., hereinafter may also be referred to as “MOI-EG”) (14.94 g), and the raw material alcohol was changed to 2-methoxyethanol (product of FUJIFILM Wako Pure Chemical Corporation) (6.28 g), to thereby yield 6-oxo-2,5,10-trioxa-7-azadodecan-12-yl methacrylate (hereinafter may also be referred to as “MEOCNMA-EG”).

### 2. Production and Analyses of Polymers

Polymers were produced according to the following Production Examples 1 to 9 and Comparative Production Examples 1 to 3. The weight average molecular weight (Mw), glass transition temperature in a dry state (Tg1), glass transition temperature in a saturated hydration state (Tg2), and saturated water content of each of the produced polymers were determined through the following methods.

### <Measurement of Weight Average Molecular Weight (Mw) of Polymer>

The weight average molecular weight (Mw) of each polymer was measured through gel permeation chromatographic (GPC) analysis under the following measurement conditions. (Measurement conditions for GPC analysis)
Apparatus: HLC-8320GPC (product of Tosoh Corporation)
Detector: RI detector
Column: TSKgel SuperMultiporeHZ-M (product of Tosoh Corporation). Three columns were connected.
Column temperature: 40° C.
Eluent: tetrahydrofuran (containing 0.03% sulfur as internal standard)
Flow rate: 350 ML/min
Calibration curve: polystyrene standard

### <Measurement of Glass Transition Temperature in a Dry State (Tg1) of Polymer>

Each of the produced polymers was dried overnight at 60° C. under reduced pressure (1,000 Pa). The thus-dried polymer was placed in an aluminum pan and weighed. Subsequently, the polymer was subjected to a heating/cooling operation by means of a differential scanning calorimeter (apparatus: DSC214Polyma, product of NETZSCH, measurement atmosphere: air). The operation profile included cooling from 100° C. to −80° C. at a temperature change rate of 5° C./min; maintaining at −80° C. for 5 minutes; and heating to 100° C. Through that operation, the glass transition temperature in a dry state (Tg1) of the polymer was determined.

### <Measurement of Glass Transition Temperature in a Saturated Hydration State (Tg2) of Polymer>

Each polymer was immersed in a large excess amount of pure water (10 g of pure water with respect to 30 mg of polymer) and allowed to stand at room temperature (25° C.) for 3 days, to thereby hydrate the polymer. The thus-hydrated polymer was picked up with tweezers from pure water, and water adsorbed on the surface of the hydrated polymer was removed by use of drug packing paper. Thereafter, the hydrated polymer (0.003 to 0.005 g) was placed in an aluminum pan and weighed. The measured weight was defined as “X (unit: g)”. Then, through a similar procedure as employed in the measurement of the glass transition temperature in a dry state (Tg1) of a polymer, the polymer was subjected to a heating/cooling operation by means of the same differential scanning calorimeter. The operation profile included cooling from 40° C. to −100° C. at a temperature change rate of 5° C./min; maintaining at −100° C. for 5 minutes; and heating to 40° C. Through that operation, the glass transition temperature in a saturated hydration state (Tg2) of the polymer was determined. The hydration state of the polymer where the top of the endothermic peak attributed to melting of ice emerges at 0° C. in a DSC curve obtained when the polymer which has been hydrated is heated by means of a differential scanning calorimeter (DSC) at a rate of 5° C./minute was defined as a “saturated hydration state”. From the DSC curve in which the top of the endothermic peak attributed to melting of ice emerges at 0° C., the glass transition temperature in a saturated hydration state (Tg2) of a polymer was determined.

### <Measurement of Saturated Water Content>

After completion of the differential scanning calorimetry, the aluminum pan was bored, and the contents were dried at 110° C. in vacuum (1 Pa) for 4 days. The change in mass before and after drying was employed as the hydration water content of the polymer (referred to simply as a “hydration water content of the polymer (unit: g)”). When the hydration water content of the polymer in a saturated hydration state was defined as “saturated hydration water content”, the relative saturated hydration water content (unit: mass %) with respect to the measured weight (X; unit: g)) was calculated by the following numerical formula (1):

$$( saturated hydration water content : mass \% ) = ( amount of \\ hydration water in polymer : g ) / X \times 100. \qquad ( 1 )$$

### Production Example 1: Production of polymer A

To a two-way branched test tube, MOCNA (3 g) serving as a monomer, 2,2′-azobis(2,4-dimethylvaleronitrile) (product of FUJIFILM Wako Pure Chemical Corporation, hereinafter may also be referred to as “initiator V-65”) (0.272 g) serving as a radical initiator, and acetonitrile (12 g) serving as a solvent were added. Then, a stirrer was put into the test tube, and a thermometer was attached to the side tube, and a three-way cock was attached to the main tube. A syringe needle was inserted through the cock, and argon was fed through the needle to the solution at 100 mL/min for 30 minutes, to thereby deoxygenate the solution. Thereafter, the test tube was closed by closing the three-way cock. The test tube was inserted into a heat block maintained at 60° C., to thereby initiate polymerization. The temperature of the heat block was appropriately adjusted so that the inside temperature of the heat block was maintained at 60° C. Three hours after start of polymerization, the test tube was cooled in an ice bath, to thereby terminate polymerization. Hexane and acetone were mixed at a mass ratio of 3:7, to thereby prepare a solvent for purification by re-precipitation. By use of the prepared solvent, purification of the reaction mixture by re-precipitation was conducted twice. Then, by use of pure water as a solvent, the recovered polymer was subjected to purification by re-precipitation, to thereby yield a polymer A. The weight average molecular weight of the polymer A, as determined through GPC measurement, was 71,300. The glass transition temperature of the polymer A in a dry state was 7° C., and the glass transition temperature in a saturated hydration state was −30° C. Thus, the difference in glass transition temperature between the saturated hydration state and dry state (ΔTg) was 37° C. The saturated water content was found to be 12.0 mass %.

### Production Example 2: Production of Polymer B

The procedure of Production Example 1 was repeated, except that there were used MEOCNA (3 g) serving as a monomer, 2,2′-azobis(2-methylbutyronitrile) (product of FUJIFILM Wako Pure Chemical Corporation, hereinafter may also be referred to as “initiator V-59”) (0.358 g) serving as a radical initiator, ethyl acetate (27 g) serving as a solvent, and a hexane-acetone (4:6 by mass) mixture serving as a solvent for purification by re-precipitation, to thereby yield a polymer B. The weight average molecular weight of the polymer B, as determined through GPC measurement, was 100, 800. The glass transition temperature of the polymer B in a dry state was-10° C., and the glass transition temperature in a saturated hydration state was −60° C. Thus, the difference in glass transition temperature between the saturated hydration state and dry state (ΔTg) was 50° C. The saturated water content was found to be 31.5 mass %.

### Production Example 3: Production of Polymer C

The procedure of Production Example 1 was repeated, except that there were used MEOCNA (7.5 g) serving as a monomer, initiator V-65 (0.003 g) serving as a radical initiator, ethyl acetate (11.2 g) serving as a solvent, and a hexane-acetone (5:5 by mass) mixture serving as a solvent for purification by re-precipitation, and polymerization was terminated 5 hours after start of polymerization, to thereby yield a polymer C. The weight average molecular weight of the polymer C, as determined through GPC measurement, was 710, 300. The glass transition temperature of the polymer C in a dry state was −11° C., and the glass transition temperature in a saturated hydration state was −60° C. Thus, the difference in glass transition temperature between the saturated hydration state and dry state (ΔTg) was 49° C. The saturated water content was found to be 30.0 mass %.

### Production Example 4: Production of Polymer D

The procedure of Production Example 1 was repeated, except that there were used MEOCNA (1.8 g) serving as a monomer, 2-methoxyethyl acrylate (hereinafter may also be referred to as “MEA”) (1.2 g), initiator V-65 (0.174 g) serving as a radical initiator, ethyl acetate (12 g) serving as a solvent, and a hexane-acetone (5:5 by mass) mixture serving as a solvent for purification by re-precipitation, to thereby yield a polymer D. The weight average molecular weight of the polymer D, as determined through GPC measurement, was 132,300. The glass transition temperature of the polymer D in a dry state was −22° C., and the glass transition temperature in a saturated hydration state was 62° C. Thus, the difference in glass transition temperature between the saturated hydration state and dry state (ΔTg) was 40° C. The saturated water content was found to be 22.4 mass %.

### Production Example 5: Production of Polymer E

The procedure of Production Example 1 was repeated, except that there were used MEOCNA (0.9 g) serving as a monomer, MEA (2.1 g), initiator V-65 (0.174 g) serving as a radical initiator, ethyl acetate (12 g) serving as a solvent, and a hexane-acetone (5:5 by mass) mixture serving as a solvent for purification by re-precipitation, to thereby yield a polymer E. The weight average molecular weight of the polymer E, as determined through GPC measurement, was 114,000. The glass transition temperature of the polymer E in a dry state was −30° C., and the glass transition temperature in a saturated hydration state was −61° C. Thus, the difference in glass transition temperature between the saturated hydration state and dry state (ΔTg) was 31° C. The saturated water content was found to be 14.5 mass %.

### Production Example 6: Production of Polymer F

The procedure of Production Example 1 was repeated, except that there were used MEOCNA (1.8 g) and butyl acrylate (hereinafter may also be referred to as “BA”) (1.2 g) serving as monomers, initiator V-65 (0.174 g) serving as a radical initiator, ethyl acetate (12 g) serving as a solvent, and a hexane-acetone (6:4 by mass) mixture serving as a solvent for purification by re-precipitation, to thereby yield a polymer F. The weight average molecular weight of the polymer F, as determined through GPC measurement, was 125, 200. The glass transition temperature of the polymer F in a dry state was 23° C., and the glass transition temperature in a saturated hydration state was −42° C. Thus, the difference in glass transition temperature between the saturated hydration state and dry state (ΔTg) was 19° C. The saturated water content was found to be 7.7 mass %.

### Production Example 7: Production of Polymer G

The procedure of Production Example 1 was repeated, except that there were used DMPOCNA (3 g) serving as a monomer, initiator V-65 (0.114 g) serving as a radical initiator, ethyl acetate (12 g) serving as a solvent, and a hexane-acetone (6:4 by mass) mixture serving as a solvent for purification by re-precipitation, to thereby yield a polymer G. The weight average molecular weight of the polymer G, as determined through GPC measurement, was 96,300. The glass transition temperature of the polymer G in a dry state was-10° C., and the glass transition temperature in a saturated hydration state was −58° C. Thus, the difference in glass transition temperature between the saturated hydration state and dry state (ΔTg) was 48° C. The saturated water content was found to be 23.4 mass %.

### Production Example 8: Production of Polymer H

The procedure of Production Example 1 was repeated, except that there were used DEPOCNA (3 g) serving as a monomer, initiator V-65 (0.104 g) serving as a radical initiator, ethyl acetate (12 g) serving as a solvent, and a hexane-acetone (8:2 by mass) mixture serving as a solvent for purification by re-precipitation, to thereby yield a polymer H. The weight average molecular weight of the polymer H, as determined through GPC measurement, was 104,800. The glass transition temperature of the polymer H in a dry state was-12° C., and the glass transition temperature in a saturated hydration state was −46° C. Thus, the difference in glass transition temperature between the saturated hydration state and dry state (ΔTg) was 34° C. The saturated water content was found to be 6.6 mass %.

### Production Example 9: Production of Polymer I

The procedure of Production Example 1 was repeated, except that there were used MEOCNMA-EG (4.5 g) serving as a monomer, initiator V-65 (0.029 g) serving as a radical initiator, ethyl acetate (10.5 g) serving as a solvent, and a hexane-acetone (9:1 by mass) mixture serving as a solvent for purification by re-precipitation, to thereby yield a polymer I. The weight average molecular weight of the polymer I, as determined through GPC measurement, was 135,100. The glass transition temperature of the polymer I in a dry state was-18° C., and the glass transition temperature in a saturated hydration state was −50° C. Thus, the difference in glass transition temperature between the saturated hydration state and dry state (ΔTg) was 32° C. The saturated water content was found to be 31.9 mass %.

### Comparative Production Example 1: Production of Polymer J

The procedure of Production Example 1 was repeated, except that there were used EOCNA (3 g) serving as a monomer, initiator V-59 (0.332 g) serving as a radical initiator, ethyl acetate (27 g) serving as a solvent, and a hexane-acetone (6:4 by mass) mixture serving as a solvent for purification by re-precipitation, to thereby yield a polymer J. The weight average molecular weight of the polymer J, as determined through GPC measurement, was 100, 400. The glass transition temperature of the polymer J in a dry state was 2° C., and the glass transition temperature in a saturated hydration state was −20° C. Thus, the difference in glass transition temperature between the saturated hydration state and dry state (ΔTg) was 22° C. The saturated water content was found to be 5.7 mass %.

### Comparative Production Example 2: Production of Polymer K

The procedure of Production Example 1 was repeated, except that there were used IPOCNA (3 g) serving as a monomer, 2,2′-azobis(2-methylbutyronitrile) (product of FUJIFILM Wako Pure Chemical Corporation) (0.358 g) serving as a radical initiator, ethyl acetate (27 g) serving as a solvent, and a hexane-acetone (7:3 by mass) mixture serving as a solvent for purification by re-precipitation, to thereby yield a polymer K. The weight average molecular weight of the polymer K, as determined through GPC measurement, was 93, 200. The glass transition temperature of the polymer K in a dry state was 18° C., and the glass transition temperature in a saturated hydration state was −8° C. Thus, the difference in glass transition temperature between the saturated hydration state and dry state (ΔTg) was 26° C. The saturated water content was found to be 4.5 mass %.

### Comparative Production Example 3: Production of Polymer L

The procedure of Production Example 1 was repeated, except that there were used MEA (3 g) serving as a monomer, initiator V-59 (0.555 g) serving as a radical initiator, ethyl acetate (27 g) serving as a solvent, and a hexane-acetone (6:4 by mass) mixture serving as a solvent for purification by re-precipitation, to thereby yield a polymer L. The weight average molecular weight of the polymer L, as determined through GPC measurement, was 84, 000. The glass transition temperature of the polymer L in a dry state was-37° C., and the glass transition temperature in a saturated hydration state was −60° C. Thus, the difference in glass transition temperature between the saturated hydration state and dry state (ΔTg) was 23° C. The saturated water content was found to be 8.4 mass %.

### 3. Production and Evaluation of Medical Coating Agent

### Examples 1 to 9 and Comparative Examples 1 to 3

By use of each of the polymers produced in Production Examples 1 to 9 and Comparative Production Examples 1 to 3, a polymer solution of a medical coating agent was produced. The medical coating agent was subjected to a fibrinogen adsorption amount test (MicroBCA assay) and a thrombocyte adsorption test. These evaluation tests were conducted through the following specific procedures.

### <Fibrinogen Adsorption Amount Test (MicroBCA Assay)>

A solution of each polymer at a concentration of 0.2 (w/v) % was prepared (i.e., acetone solution (Example 1), as were ethyl acetate solution (Comparative Examples 1 and 2), and methanol solution (Examples 2 to 9 and Comparative Example 3), to thereby provide a medical coating agent. Each medical coating agent (15 μL) was added dropwise to each well of a 96-well plate (product of Corning; general assay plate, made of polypropylene, flat 96-well perfect plate, non-sterilized) and allowed to stand for 3 days to dry the agent. Thus, a coating substrate for evaluation (hereinafter referred to as evaluation coating substrate) 1 was provided. Separately, fibrinogen was dissolved in PBS (−) (product of FUJIFILM Wako Pure Chemical Corporation) to a concentration of 1 mg/mL, and the solution was added to the well in an amount of 50 ML/well. Then, cultivation was conducted at 37° C. for 10 minutes. After cultivation, liquid remaining in the wells was removed, and the wells were washed with PBS (−) in an amount of 200 μL/well. The washing operation was repeated 7 times, and the wells were dried. Thereafter, an extraction liquid (i.e., a solution prepared by mixing 5% aqueous SDS with 0.1N aqueous sodium hydroxide (1:1 by volume)) was added to the wells in an amount of 50 UL/well. Then, cultivation was conducted at 37° C. for 2 hours.

After cultivation, PBS (−) (50 UL/well) and a working reagent (100 μL) were added to each well. The working reagent was prepared according to the instruction manual of Micro BCA Protein Assay Kit (product of Thermo Scientific). Subsequently, the plate was heated at 60° C. for 1 hour. After completion of heating, absorbance at 540 nm was measured by means of a plate reader (Vmax KINETIC MICROPLATE READER, product of FUJIFILM Wako Pure Chemical Corporation). On the basis of the fibrinogen concentration of the resultant extraction liquid, the amount of fibrinogen adsorbed in a unit area of the evaluation coating substrate 1 (μg/cm²) (hereinafter may also be referred to as “FIB adsorption amount”) was calculated. Conceivably, the smaller the value, the more excellent the anti-thrombogenicity. A calibration curve for calculating the concentration was drawn by use of bovine serum albumin attached to Micro BCA Protein Assay Kit (product of Thermo Scientific) according to the instruction manual of the kit.

### <Thrombocyte Adsorption Test>

A solution of each polymer at a concentration of 0.2 (w/v) % was prepared (i.e., acetone solution (Example 1), as were ethyl acetate solution (Comparative Examples 1 and 2), and methanol solution (Examples 2 to 9 and Comparative Example 3), to thereby provide a medical coating agent. Separately, polyethylene terephthalate (PET) sheet (“DIAFOILT-100E,” product of Mitsubishi Chemical; size: 5 cm×5 cm; thickness: 125 μm) was sufficiently washed with acetone. Each medical coating agent (650 UL) was applied onto the PET sheet through spin coating. The spin coating profile was 500 rpm for 5 s→1,500 rpm for 10 s→1,500 to 4,000 rpm (slope) for 5 s→4,000 rpm for 10 s→4,000 to 0 rpm (slope) for 5 s. Thereafter, the sheet was dried at room temperature (25° C.) for 3 days, to thereby provide an evaluation coating substrate 2 with respect to each medical coating agent.

The thus-obtained evaluation coating substrate 2 was cut into pieces (8 mm×8 mm). Separately, thrombocytes were added to blood plasma, to thereby provide a plasma liquid having an inoculation thrombocyte concentration of 4×10⁷ cells/cm². The plasma liquid (200 μL) was placed on each coating substrate piece (i.e., evaluation coating substrate 2). Culturing was conducted at 37° C. for 1 hour. Then, the pieces of the evaluation coating substrate 2 were washed twice with PBS (−). The washed substrate pieces were immersed in 1% glutaraldehyde PBS (−) solution and allowed to stand overnight at 4° C. The pieces of the evaluation coating substrate 2 were removed from the solution and washed sequentially with PBS (−), an aqueous solution prepared by mixing PBS (−) with water at a volume ratio of 1:1, and pure water. Before each washing operation, each piece of the evaluation coating substrate 2 was immersed in the corresponding washing liquid for 10 minutes.

After the washing operation, each piece of the evaluation coating substrate 2 was air-dried at room temperature (25° C.) for 3 days. The number of thrombocytes adsorbed on the piece of the evaluation coating substrate 2 was counted through observation under a scanning electron microscope (JSM-7900F, product of JEOL Ltd.; vacuum degree: 30 Pa; acceleration voltage: 15 kV). Counting of each piece was conducted in 5 vision fields (magnification: 1,500, 4.8×10⁻⁵ cm²). The number of thrombocytes observed in one vision field was counted, and the counts of the 5 vision fields were averaged, to thereby provide a thrombocyte adsorption number (cells/field). The smaller the number, the more excellent the anti-thrombogenicity.

### Comparative Example 4

The same fibrinogen adsorption test and thrombocyte adsorption test conducted in Examples 1 to 9 and Comparative Examples 1 to 3 were performed, except that no coating treatment with the medical coating agent was performed to a 96-well plate or a PET sheet.

Table 1 shows the characteristics of the polymers employed in Examples 1 to 9 and Comparative Examples 1 to 3, evaluation results of the medical coating agents, and evaluation results of Comparative Example 4.

TABLE 1

| | Polymer | | | | | | | Evaluation results | |
|---|---|---|---|---|---|---|---|---|---|
| | | | | Tg in dry | Tg in saturated | | Saturated water | | Thrombocyte |
| | Polymer | | | state (Tg1) | hydration state | ΔTg | content | FIB adsorption | adsorption number |
| | name | Monomer | Mw | [° C.] | (Tg2) [° C.] | [° C.] | [mass %] | amount | [cells/field] |
| Example 1 | Polymer | MOCNA | 71.300 | 7 | −30 | 37 | 12.0 | 6.9 | 16 |
| | A | | | | | | | | |
| Example 2 | Polymer | MEOCNA | 100.800 | −10 | −60 | 50 | 31.5 | 2.1 | 5 |
| | B | | | | | | | | |
| Example 3 | Polymer | MEOCNA | 710.300 | −11 | −60 | 49 | 30.0 | 1.9 | 7 |
| | C | | | | | | | | |
| Example 4 | Polymer | MEOCNAMEA | 132.300 | −22 | −62 | 40 | 22.4 | 2.7 | 4 |
| | D | (6/4 w/w) | | | | | | | |
| Example 5 | Polymer | MEOCNA/MEA | 114.000 | −30 | −61 | 31 | 14.5 | 6.2 | 14 |
| | E | (3/7 w/w) | | | | | | | |
| Example 6 | Polymer | MEOCNA/BA | 125.200 | −23 | −42 | 19 | 7.7 | 5.6 | 12 |
| | F | (6/4 w/w) | | | | | | | |
| Example 7 | Polymer | DMPOCNA | 96.300 | −10 | −58 | 48 | 23.4 | 1.5 | 4 |
| | G | | | | | | | | |
| Example 8 | Polymer | DEPOCNA | 104.800 | −12 | −46 | 34 | 6.6 | 6.4 | 16 |
| | H | | | | | | | | |
| Example 9 | Polymer | MEOCNMA-EG | 135.100 | −18 | −50 | 32 | 31.9 | 3.4 | 6 |
| | I | | | | | | | | |
| Comparative | Polymer | EOCNA | 100.400 | 2 | −20 | 22 | 5.7 | 13.3 | 42 |
| Example 1 | J | | | | | | | | |
| Comparative | Polymer | IPOCNA | 93.200 | 18 | −8 | 26 | 4.5 | 15.0 | 50 |
| Example 2 | K | | | | | | | | |
| Comparative | Polymer | MEA | 84.000 | −37 | −60 | 23 | 8.4 | 8.3 | 22 |
| Example 3 | L | | | | | | | | |
| Comparative | | | | | | | | | |

| | | | |
|---|---|---|---|
| Example 4 | No coating treatment | 17.5 | 57 |

### 4. Results of Evaluation

As is clear from Table 1, the medical coating agents each containing a polymer including the structural unit (A) and having a glass transition temperature in a saturated hydration state (Tg2) of −25° C. or lower were found to provide a small fibrinogen adsorption amount (FIB adsorption amount) and a small thrombocyte adsorption number; i.e., showing excellent anti-thrombogenicity. The polymers A to I employed in Examples 1 to 9 had a hydrated water content of 6% or more. Thus, conceivably, intermediate water was localized in the surface of the polymers, which impeded recognition of the substrate as a foreign object by a biocomponent, leading to excellent anti-thrombogenicity.

In contrast, the medical coating agents of Comparative Examples 1 and 2, each containing the polymer J or K including the structural unit (A) and having a glass transition temperature in a saturated hydration state (Tg2) higher than −25° C., were found to provide a larger fibrinogen adsorption amount (FIB adsorption amount) and a greater thrombocyte adsorption number, as compared with the medical coating agents of Examples 1 to 9. Thus, the anti-thrombogenicity was insufficient. Also, the medical coating agent of Comparative Example 3, containing the polymer L including no structural unit (A) and including an MEA unit, were found to provide a larger fibrinogen adsorption amount (FIB adsorption amount) and a greater thrombocyte adsorption number, as compared with the medical coating agents of Examples 1 to 9. Thus, the anti-thrombogenicity was poor.

The present invention is not limited to the aforementioned embodiments. Needless to say, the present invention encompasses various modifications and those falling within the equivalents thereof, so long as they are not deviated from the gist of the present invention. Thus, it should be construed that, in view of the above teaching, those skilled in the art could conceive various combinations, modes, and further embodiments of a single element or a combination including the element or its equivalent, which also fall within the scope or concept of the present invention.

1. A medical coating agent comprising a polymer comprising a structural unit (A) derived from an ethylenically unsaturated monomer having a urethane bond,
wherein the polymer has a glass transition temperature of −25° C. or lower in a saturated hydration state, the saturated hydration state being defined as such a hydration state of the polymer that the top of the endothermic peak attributed to melting of ice emerges at 0° C. in a DSC curve obtained when the polymer which has been hydrated is heated by means of a differential scanning calorimeter (DSC) at a rate of 5° C./minute.

2. The medical coating agent according to claim 1, wherein the polymer comprises the structural unit (A) in an amount of 10 mass % or more with respect to the entire structural units comprised in the polymer.

3. The medical coating agent according to claim 1, wherein the polymer exhibits a difference in glass transition temperature of 25° C. or greater between a dry state and the saturated hydration state, the glass transition temperatures being determined by means of differential scanning calorimeter at a temperature elevation rate of 5° C./minute.

4. The medical coating agent according to claim 1, wherein the polymer is a (meth)acrylic polymer.

5. A medical device comprising:
a substrate; and
the medical coating agent according to claim 1 provided on the substrate.
